SEQUENCE LISTING (5 sequences)

SEQ ID NO: 1 - protein, 10 aa, Artificial sequence
  C-terminal epitope sequence in NC1 domain of collagen type VI alpha 1 as selection peptide
SFSGFLVAPM

SEQ ID NO: 2 - protein, 10 aa, Artificial sequence
  Modified C-terminal epitope sequence as a screening peptide
SFSGFLVAPM

SEQ ID NO: 3 - protein, 8 aa, Artificial sequence
  Truncated peptide
SFSGFLVA

SEQ ID NO: 4 - protein, 13 aa, Artificial sequence
  Modified C-terminal epitope sequence as an immunogenic peptide
CGGSFSGFLVAPM

SEQ ID NO: 5 - protein, 11 aa, Artificial sequence
  Deselection peptide
DMDYLPRVPNQ

(19) United States
(12) Patent Application Publication
(10) Pub. No.: US 2016/0123993 A1
(43) Pub. Date: May 5, 2016

(54) Collagen Type X Alpha-1 Assay

(71) Applicant: NORDIC BIOSCIENCE A/S, Herlev (DK)

(72) Inventors: Anne-Christine Bay Jensen, Kobenhavn S (DK); Yi He, Ballerup (DK); Morten Karsdal, Kobenhavn O (DK)

(73) Assignee: Nordic Bioscience A/S, Herlev (DK)

(21) Appl. No.: 14/889,905

(22) PCT Filed: May 9, 2014
(86) PCT No.: PCT/EP2014/059584

§ 371 Date: Nov. 9, 2015

(30) Foreign Application Priority Data
May 10, 2013 (GB) 1308396.9

(51) Int. Cl. G01N 33/68; G01N 33/564; C07K 16/18

(52) U.S. Cl. CPC G01N 33/6893; C07K 16/18; G01N 33/564; C07K 2317/34; G01N 2333/78; G01N 2800/105; G01N 2800/10

(57) ABSTRACT

An antibody specifically reactive with an epitope of collagen type X alpha 1 comprised in the NC1 domain C-terminal amino acid sequence SFSGFLVAPM-COOH (SEQ ID NO: 1), and a method of immunoassay for detecting in a biological sample an epitope comprised in the NC1 domain C-terminal amino acid sequence SFSGFLVAPM-COOH (SEQ ID NO: 1) of collagen type X alpha 1, by contacting said biological sample with said antibody, and determining the amount of binding of said antibody.

### TECHNICAL FIELD

The present invention relates to an antibody which specifically reacts with an epitope of collagen type X alpha 1, and its use in a method of immunoassay for detecting and quantifying collagen type X alpha 1.

### BACKGROUND ART

Osteoarthritis (OA) is a common joint disease which is characterized by cartilage damage and loss of joint function. The etiology of OA comprises multiple factors including aging, obesity, trauma and heredity [1]. The pathogenesis of OA is poorly understood due to the heterogeneity and complexity of this disease.

Remarkably, some characteristics of OA resemble chondrocyte differentiation processes during skeletal development by endochondral ossification. In healthy articular cartilage, chondrocytes resist proliferation and terminal differentiation. By contrast, chondrocytes in diseased cartilage progressively proliferate and develop hypertrophy.

Moreover, vascularization and focal calcification of joint cartilage are initiated [2-5]. The molecular events regulating chondrocyte differentiation are still unknown, but chondrocyte hypertrophy-like changes in OA have attracted more attention for study [6-8}.

### Type X Collagen Alpha-1

Collagen type X alpha-1 is non-fibrillar, but forms fine pericellular filaments in association with cartilage collagen. The molecule isolated from chondrocyte cultures or from cartilage is a homotrimer of 59 kDa Collagen type X alpha-1 chains, and there have been reports of a recombinant molecule of collagen type X of approximately 75 kDa [9]. Collagen type X alpha-1 shares a similar domain structure with type VIII collagen: a central triple-helical (COLI) domain of 50 kDa is flanked by N-terminal (NC2) and C-terminal (NC1) non-triple-helical domains [10]. In addition, both collagen types represent major components of hexagonal lattice structure, in which the collagen molecules link together by interactions involving the non-triple-helical end regions.

Collagen type X alpha-1 distribution is restricted to normal fetal hypertrophic cartilage in the growth zones of long bones, vertebrae and ribs, and in adult (>21 yr) thyroid cartilage, where it may provide a scaffold to prevent local collapse as the cartilage matrix is removed during endochondral ossification [11]. It is also found in bone fracture callus, in osteoarthritic cartilage and in chondrogenic neoplasms, and may be involved in cartilage mineralization.

### Osteoarthritis and Ankylosing Spondylitis

Ankylosing Spondylitis (AS) is a chronic inflammatory disease of the spine and sacroiliac joints, whereas OA is generally considered to be a non-inflammatory condition of the synovial joints, predominantly knee and hips. Chondrocyte hypertrophy and cartilage calcification are key pathological events in both joint diseases. Elevated expression of network-forming type X collagen is believed to be a specific signal for chondrocyte hypertrophy [12-15] therefore type X collagen can be used as a detectable marker for said diseases.

There are several proteins associated with hypertrophic chondrocytes, such as collagen type X, MMP13, osteopontin, osteocalcin [16], Indian Hedgehog [17], Runx2 [18], VEGF [19], HtrA1 [20] and Transglutaminase-2 (TG-2)[21]. Collagen type X and MMP13 are among the most widely used as markers of hypertrophic chondrocytes. However, synthesis of MMP13 can be induced in chondrocytes by inflammation and mechanical stress [22-23]. Therefore, collagen type X as a hypertrophic chondrocyte specific marker can indicate a phenotype alteration of chondrocytes.

Thus, a method which accurately quantifies the amount of collagen type X or its fragments in a biological sample may allow a better understanding of collagen type X pathologies or physiological processes affecting collagen type X turnover such as OA or AS. Evidently there is a need for such a method.

### DESCRIPTION OF THE INVENTION

The herein described invention relates to an antibody directed to an epitope of collagen type X alpha 1 in the NC1 domain C-terminal amino acid sequence SFSGFLVAPM-COOH (SEQ ID NO: 1) and a method of immunoassay for detecting and quantifying the amount of intact collagen type X alpha 1 and fragments thereof comprising the amino acid sequence SFSGFLVAPM-COOH (SEQ ID NO: 1).

In a first aspect, the present invention relates to an antibody, wherein said antibody specifically reacts with an epitope of collagen type X alpha 1, said epitope being comprised in the NC1 domain C-terminal amino acid sequence SFSGFLVAPM-COOH (SEQ ID NO: 1).

In a preferred embodiment of the invention, said antibody is a monoclonal antibody, or a polyclonal antibody, or an antibody fragment.

In a preferred embodiment of the invention, said antibody specifically reacts with an epitope comprised in the NC1 domain C-terminal amino acid sequence SFSGFLVAPM (SEQ ID NO: 1) of human collagen type X alpha 1.

It should be understood that the antibody of the present invention is an artificial product resulting from the selection of a particular antigenic sequence determined by computational means (e.g. BLAST analysis) and generated by an artificially induced immune response. It should be understood that said antibody is not a product that has been isolated from a source that occurs naturally in nature.

In another aspect, the present invention relates to a method of immunoassay for detecting in a biological sample an epitope comprised in the NC1 domain C-terminal amino acid sequence SFSGFLVAPM-COOH (SEQ ID NO: 1) of collagen type X alpha 1, said method comprising contacting said biological sample comprising said epitope comprised in said NC1 domain C-terminal amino acid sequence SFSGFLVAPM-COOH (SEQ ID NO: 1) of collagen type X alpha 1 with an antibody of the invention, and determining the amount of binding of said antibody.

In a preferred embodiment of the invention, said method of immunoassay is used to quantify the amount of intact collagen type X alpha 1 and fragments thereof comprising the amino acid sequence SFSGFLVAPM (SEQ ID NO: 1) in biofluid, wherein said biofluid may be, but is not limited to, synovial fluid, serum or plasma.

In a preferred embodiment of the invention, said method of immunoassay may be, but is not limited to, a competition assay or a sandwich assay.

In a preferred embodiment of the invention, said method of immunoassay may be, but is not limited to, a radioimmunoassay or an enzyme-linked immunosorbent assay.

It should be understood that intact collagen type X alpha 1 and fragments thereof comprising the amino acid sequence SFSGFLVAPM can be detected and quantified using assay methods other than that of the present invention. Such methods include quantitative chromatographic techniques, 1D- and 2D-electrophoresis techniques, and quantitative mass spectrometry.

In a preferred embodiment of the invention, said method of immunoassay further comprises correlating the quantity of intact collagen type X alpha 1 and fragments thereof comprising said amino acid sequence SFSGFLVAPM (SEQ ID NO: 1) determined by said method with standard disease samples of known disease severity to evaluate the severity of a disease associated with collagen type X alpha 1.

In a preferred embodiment of the invention, said method of immunoassay further comprises correlating the quantity of intact collagen type X alpha 1 and fragments thereof comprising said amino acid sequence SFSGFLVAPM (SEQ ID NO: 1) determined by said method with standard Osteoarthritis samples of known severity.

In a preferred embodiment of the invention, said method of immunoassay further comprises correlating the quantity of intact collagen type X alpha 1 and fragments thereof comprising said amino acid sequence SFSGFLVAPM (SEQ ID NO: 1) determined by said method with standard Ankylosing Spondylitis samples of known severity.

Preferably, the present invention relates to a method for evaluating the severity of a disease associated with collagen type X alpha 1 in a human patient, such as Osteoarthritis or Ankylosing Spondylitis, said method comprising:
obtaining a biological sample from a patient;
contacting said biological sample with an antibody of the invention;
determining the amount of binding of said antibody using either a radioimmunoassay or an enzyme-linked immunosorbent assay, thereby quantifying the amount of intact collagen type X alpha 1 and fragments thereof comprising the amino acid sequence SFSGFLVAPM (SEQ ID NO: 1) in said biological sample;
correlating the quantity of intact collagen type X alpha 1 and fragments thereof comprising said amino acid sequence SFSGFLVAPM (SEQ ID NO: 1) with standard samples of a disease associated with collagen type X alpha 1, such as Osteoarthritis or Ankylosing Spondylitis; and
determining the severity of the disease associated with collagen type X alpha 1, such as Osteoarthritis or Ankylosing Spondylitis, in said patient.

In another aspect, the present invention relates to an assay kit for determining the quantity of intact collagen type X alpha 1 and fragments thereof comprising the amino acid sequence SFSGFLVAPM (SEQ ID NO: 1) in a biological sample, said kit comprising an antibody as described herein and at least one of:
a streptavidin coated 96 well plate
a biotinylated peptide Biotin-L-SFSGFLVAPM-COOH (SEQ ID NO: 2), wherein L is an optional linker
a secondary antibody for use in a sandwich immunoassay
a calibrator peptide comprising the sequence SFSGFLVAPM (SEQ ID NO: 1)
an antibody biotinylation kit
an antibody HRP labeling kit
an antibody radiolabeling kit
an assay visualization kit

### DEFINITIONS

“Antibody” as used herein refers to a monoclonal antibody, a polyclonal antibody, or an antibody fragment, such as Fab, F(ab′)₂, Fv, or scFv fragments etc., or a chemically modified derivative of any of these.

“C-Col10” is used to distinguish the herein described collagen type X assay from the collagen type X assays known in the art which are not based on the specific binding of epitopes comprised within the amino acid sequence SFSGFLVAPM.

### FIGURES

FIG. 1: Antibody specificity evaluated by two synthetic peptides: selection peptide (SFSGFLVAPM (SEQ ID NO: 1)) and truncated peptide (SFSGFLVA (SEQ ID NO: 3)).

FIG. 2: Western blotting of U2-OS cell lysates. Lane 1, 4, and 9: U2-OS cell lysates; Lane 2, 5, 7 and 10: RIPA buffer; Lane 3 and 8: Molecular weight standard.

### EXAMPLES

### Materials and General Considerations

All reagents used in the experiments were high-standard chemicals from companies such as VWR (Rødovre, Denmark) and Sigma Aldrich (Brøndby, Denmark). The synthetic peptides used for monoclonal antibody production and validation were 1) Immunogenic peptide: KLH-CGG-SFSGFLVAPM-COOH (KLH=Keyhole Limpet Hemocyanin)(SEQ ID NO: 4) 2) Screening peptide: Biotin-SFSGFLVAPM-COOH (SEQ ID NO: 2), and 3) Selection peptide: SFSGFLVAPM-COOH (SEQ ID NO: 1). All synthetic peptides were purchased from Thermo Fisher, Beijing, China.

### Example 1

### Monoclonal Antibody Generation—NB509-11G8

The sequence for the C-terminal NC1 domain type X collagen was selected from homology between species and uniqueness among other ECM proteins by protein blasting. It was directed against the C-terminal NC1 domain and selected for minimization of homology to other human proteins and optimization of immunogenicity. The resulting epitope amino acid sequence was SFSGFLVAPM-COOH (SEQ ID NO: 1). Generation of monoclonal antibodies was initiated by subcutaneous immunization of 6 week old Balb/C mice with 200 μl emulsified antigen (Freund's adjuvant) and 60 μg of the collagen type X alpha 1 epitope sequence (KLH-CGG-SFSGFLVAPM-COOH (SEQ ID NO: 4)). Two further immunizations of 30 μg immunogen in 200 μl emulsified antigen were given 2 weeks apart and four final immunizations of 30 μg immunogen in 200 μl emulsified antigen were given 3 weeks apart. Blood samples were collected from the 3rd immunization. Each sample was stored at −20° C. prior to analysis. At each blood sampling, the serum titer was determined and the mouse with highest antiserum titer was selected for fusion. After the final immunization, this mouse was rested for 1 month and then boosted intravenously with 50 μg immunogen in 100 μl 0.9% sodium chloride solution three days before isolation of the spleen for cell fusion. The fusion procedure was performed as described by Gefter et al [24]. Briefly, mouse spleen cells were fused with SP2/0 myeloma fusion partner cells. The hybridoma cells were cloned using a semi-solid medium method and transferred into 96-well microtiter plates for further growth and incubated in a CO2-incubater. Standard limited dilution was used to promote monoclonal growth. The supernatants were screened for reactivity and selectivity against the calibrator peptide (SFSGFLVAPM (SEQ ID NO: 1)), deselection peptide (DMDYLPRVPNQ (SEQ ID NO: 5)) and truncated peptide (SFSGFLVA (SEQ ID NO: 3)). Biotin-SFSGFLVAPM-COOH (SEQ ID NO: 2) was used as screening peptide. The isotype of the monoclonal antibodies was determined using the Clonotyping System-HRP kit, cat. 5300-05 (Southern Biotech, Birmingham, Ala., USA).

### Clone Characterisation

Human osteosarcoma cell lines (U2-OS; collagen X producing cell line) were purchased from ATCC (USA) and cultivated in DMEM medium containing 10% FBS, 2 mM L-Glutamine, 100 units/ml penicillin and 100 ug/ml streptomycin. Cells were grown in T25 flasks in a 37° C. incubator at 5% CO2, changing the medium every two or three days. When the cells reached 90% confluency the cell lysates were prepared using RIPA lysis buffer with following procedure: the cell media was removed and the cells washed twice with PBS, followed by adding cold RIPA lysis buffer (25 mM Tris-HCl pH7, 6; 150 mM NaCl, 1% Sodium deoxycholate acid). An EDTA free cocktail tablet was added to the flask then RIPA buffer was distributed to cover the whole surface of flask and incubated on ice for 10 minutes. The cells were scraped and centrifuged at 10,000 RPM at 4° C. for 10 minutes and the supernatants stored at −20° C. until use.

U2-OS lysates were electrophoresed on 4-12% Bis-Tris gradient gel under reducing conditions using MES SDS running buffer. Protein bands were blotted onto a nitrocellulose membrane using the Invitrogen i-Blot gel transfer system according to manufacturer's instruction. The membrane was blocked in blocking buffer (5% skimmed milk in Tris-buffered saline with Tween (TBST)) overnight at 4° C. The monoclonal antibody, NB509-11G8 and commercial collagen type X antibody X53 (for comparison purposes) were applied in a concentration of 1 μg/ml in TBST with 5% skim milk powder and shaken overnight at 4° C. After washing 6 times with TBST, the anti-mouse secondary antibody was applied in 1:5000 in TBST with shaking at RT for 2 hours. The membrane was washed 6 times with TBST and the bands were visualized using an electro-chemiluminescence machine. To confirm the specificity of bands, a blocking western blot was performed with the same procedure, however with the addition of 3 μg/ml selection peptide (SFSGFLVAPM (SEQ ID NO: 1)) or truncated peptide (SFSGFLVA (SEQ ID NO: 3)) into the NB509-11G8 solution.

### Clone Selection and Characterization

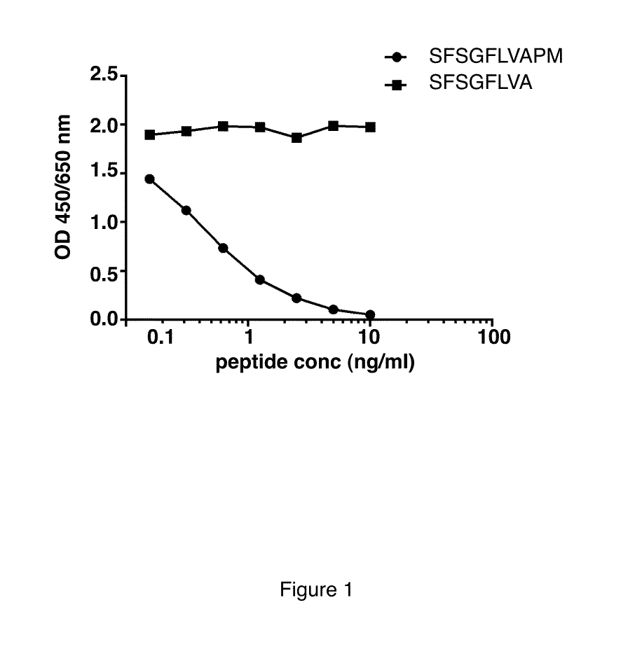

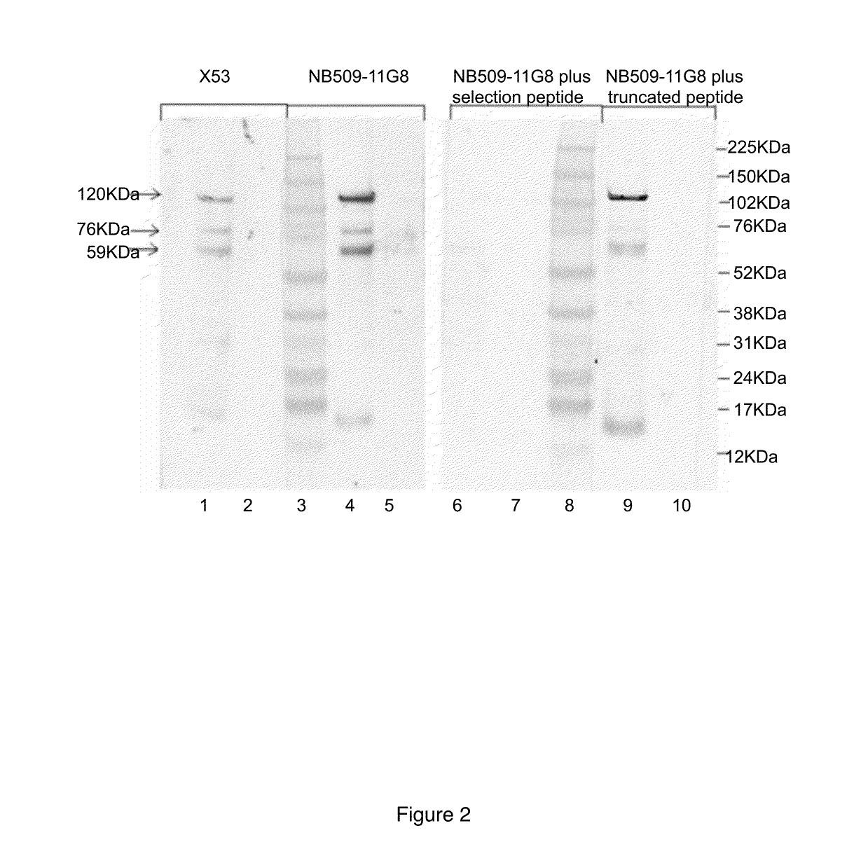

The subtype was determined to be an IgG1,k subtype. Antibody NB509-11G8 was found to be reactive with the selection peptide (SFSGFLVAPM (SEQ ID NO: 1)) whilst being non-reactive with the deselection peptide (DMDYLPRVPNQ (SEQ ID NO: 5)) and the truncated peptide (SFSGFLVA (SEQ ID NO: 3)) (no inhibition observed in the preliminary assays)(FIG. 1 shows the difference in reactivity of the antibody with the selection peptide and the truncated peptide). From the Western Blot analysis (FIG. 2) it was seen that the commercial antibody X53 and the collagen type X alpha 1 NC1 domain C-terminal epitope specific monoclonal antibody NB509-11G8 recognized three bands with molecular sizes around 59 kDa, 76 kDa and 120 kDa. The bands at 59 and 120 kDa are the collagen type X monomer and dimer, respectively, and the band at 76 kDa is assumedly the previously reported recombinant collagen type X molecule [9]. All three bands could be inhibited by the selection peptide blocking experiment, whereas no inhibition was observed whilst using the truncated peptide. A further Western blot analysis of collagen type X alpha 1 treated with collagenase demonstrated binding of NB509-11G8 to multiple fragments, confirming that NB509-11G8 binds fragments of collagen type X alpha 1 as well as the intact molecule.

### Example 2

### Enzyme-Linked Immunosorbent Assay (ELISA)

### ELISA Assay Generation/Optimization

The buffer type, coater concentration, antibody concentration and incubation conditions were optimised using standard methods.

### C-Col10 ELISA Protocol

The competitive C-Col10 ELISA procedure was as follows: A 96-well streptavidin-coated ELISA plate from Roche, cat.11940279, was coated with the biotinylated peptide Biotin-SFSGFLVAPM-COOH (SEQ ID NO: 2) dissolved in coater buffer (25 mM PBS-BTB, pH 7.4) at 4 ng/ml in 100 μl, incubated for 30 min at 20° C. in the dark and subsequently washed in washing buffer (20 mM Tris, 50 mM NaCl, pH 7.2). Thereafter μl of peptide calibrator or sample was added to the appropriate wells, followed by 100 μl of HRP-conjugated monoclonal antibody NB509-11G8 (labeled with HRP using Lightning-Link™ HRP Conjugation Kit (Innova Biosciences, Babraham, Cambridge, UK), according to the manufacturer's instructions) dissolved in incubation buffer (25 mM PBS-BTB, pH 7.4) at 190 ng/ml. The plate was incubated for 20 hours at 4° C. and washed. Finally, 100 μl tetramethylbenzinidine (TMB) (Kem-En-Tec cat.: 4380H) was added, the plate was incubated for 15 min at 20° C. in the dark and the reaction was stopped by addition of 100 μl of stopping solution (2M H₂SO₄). The plate was analyzed in an ELISA reader at 450 nm with 650 nm as the reference (Molecular Devices, SpectraMax M, CA, USA).

### Technical Evaluation of C-Col10 ELISA

The lower detection limit (LDL) was calculated from 21 determinations of the lowest standard (the zero standard) and calculated as the mean+3 standard deviation. The LDL for the assay was 0.062 ng/mL. The inter- and intra-assay variation was determined by 10 independent runs of 8 QC samples, with each run consisting of two replicas of double determinations of the samples.

The inter- and intra-assay variation was a mean 13.18% and 4.19% respectively. For each assay a master calibrator prepared from synthetic peptides accurately quantified by amino acid analysis was used for calibration purposes. The linearity-dilution of human serum is acceptable down to 1:4 and the measurement range is 2-0.088 ng/mL.

### Example 3

### Correlation Between C-Col10 and C2M

Matrix metalloprotein derived collagen type II fragment (C2M) has been shown to be a marker of cartilage degradation [25]. The C-Col10 assay significantly correlated with cartilage degradation marker measured by C2M assay (Pearson r=0.5748, P<0.0001). This suggests that hypertrophy-like changes in OA may be associated with cartilage degradation. Therefore, measurement of collagen type X as a marker of hypertrophic chondrocytes may offer an alternative method for monitoring cartilage degradation in OA.

### Example 4

### Immunolocalisation

A further use for antibody NB509-11G8 can be found in immunolocalisation studies. 5 human cartilage samples were processed using the following procedure: fixed and decalcified cartilage were embedded in paraffin wax and cut into 5 μm thick sections. Sections were melted at 60° C., deparaffinized, and hydrated. For collagen type X, antigen retrieval was performed using Pronase E (Roche) at 37° C. for 15 minutes, while sections were demarked in the citrate buffer pH 6.0 at 60° C. overnight. Unspecific binding was blocked with 0.5% casein in TBS buffer at RT for 20 minutes. Then NB509-11G8 solution or normal mouse IgG solution (negative control) were incubated with sections at 4° C. overnight (20±1 h). Immunoactivity was detected by using peroxidase labeled anti-mouse system and diaminobenzidine (DAB, Dako, Denmark) as the chromogen. Sections were counter stained with Mayer's acidic hematoxylin for 12 seconds.

The distribution of collagen type X was tested using standard immunohistochemistry methods known in the art. Collagen type X was predominately detected in the deep zone and calcified cartilage in a mild OA sample, of which the surface was uneven and showed surface fibrillation. When vertical fissures extended into the mid zone, a strong signal of collagen type X was observed in the mid zone. However, when surface erosion, cartilage lesions and clustering of chondrocytes were present, collagen type X stained the matrix around clustered chondrocytes.

### Example 5

### Study of C-Col10 in OA Patients

### Serum Samples

Serum samples were retrieved from a C4Pain study (n=271 with Kellgren-Lawrence score ranging from 0-4; briefly, the K-L score is a scoring system based on x-ray radiographs of a patient's joints, wherein the score is determined by a trained radiographer, and consists of measuring joint space narrowing, osteophytes and sclerosis). In this study, the OA population was recruited based on intensity of knee joint pain, with the patients being selected across the pain score range of from 0 to 100 on a Western Ontario and McMaster Universities Osteoarthritis Index (WOMAC) pain scale. Two plain X-ray examinations in standing position were performed. The Kellgren-Lawrence score ranging from 0-4 of participants were obtained. Serum was collected upon overnight fasting prior to surgery or during consultation. The study was approved by The Ethical Committee of Northern Jutland (VEK no.: N-20100094). They were conducted according to the Principal of Good Clinical Practice and according to the Declaration of Helsinki. All patients provided written informed consent.

### Assessment of C-Col10 in OA Serum Samples

271 subjects were stratified into 5 groups based on their KL score (Table 1). There was significant difference in the Col10 levels of KL0 and KL2 (p=0.04). The mean value of Col10 in KL3 and KL 4 groups were 1.5 and 1.7 times the mean value of Col10 in KL0 group, respectively, however, this was not significant (due to insufficient number of study participants).

The results demonstrate a general increase in Col10 compared to KL score, which shows the usefulness of using the herein described Col10 immunoassay for OA diagnostic purposes.

TABLE 1
Serum levels of Col10 in 271 samples divided by KL
score. The data is shown as mean [95%-CI]. Col10: C-terminus
of Col10 assay; KL score: Kellgren-Lawrence score. Unpaired
t-test was applied to compare to log transformed data when
compare the levels.

| KL | Number of | | | C-Col10 | p |
|---|---|---|---|---|---|
| score | Female/Male | Age | BMI | pg/ml | value |
| 0 | 4/6 | 62.5 | 25.4 | 52 | — |
| | | (57.3-67.7) | (23.7-27.0) | (24-80) | |
| 1 | 31/28 | 63.7 | 27.0 | 65 | 0.11 |
| | | (61.6-65.8) | (26.0-28.1) | (54-76) | |
| 2 | 79/65 | 64.7 | 28.2 | 86 | 0.04* |
| | | (63.5-65.9) | (27.5-28.8) | (73-98) | |
| 3 | 17/19 | 64.3 | 29.3 | 80 | 0.07 |
| | | (61.9-66.7) | (27.4-31.2) | (60-101) | |
| 4 | 12/10 | 67.8 | 29.5 | 87 | 0.28 |
| | | (64.4-71.2) | (27.8-31.2) | (47-128) | |

p value calculated with respect to KL = 0.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’. All prior teachings acknowledged above are hereby incorporated by reference. No acknowledgement of any prior published document herein should be taken to be an admission or representation that the teaching thereof was common general knowledge in Australia or elsewhere at the date hereof.

### REFERENCES

1. Abhishek, A. and M. Doherty, Pathophysiology of articular chondrocalcinosis role of ANKH. Nat Rev Rheumatol, 2011; 7:96-104.
2. Kronenberg, H. M., Developmental regulation of the growth plate. Nature, 2003; 423:332-6.
3. Pfander, D., B. Swoboda, and T. Kirsch, Expression of early and late differentiation markers (proliferating cell nuclear antigen, syndecan-3, annexin VI, and alkaline phosphatase) by human osteoarthritic chondrocytes. Am J Pathol, 2001; 159:1777-83.
4. von der Mark, K., et al., Type X collagen synthesis in human osteoarthritic cartilage. Indication of chondrocyte hypertrophy. Arthritis Rheum, 1992; 35:806-11.
5. Fuerst, M., et al., Calcification of articular cartilage in human osteoarthritis. Arthritis Rheum, 2009; 60:2694-703.
6. Dreier, R., Hypertrophic differentiation of chondrocytes in osteoarthritis: the developmental aspect of degenerative joint disorders. Arthritis Res Ther, 2010; 12:216.
7. Pitsillides, A. A. and F. Beier, Cartilage biology in osteoarthritis—lessons from developmental biology. Nat Rev Rheumatol, 2011; 7:654-63.
8. van der Kraan, P. M. and W. B. van den Berg, Chondrocyte hypertrophy and osteoarthritis: role in initiation and progression of cartilage degeneration? Osteoarthritis Cartilage, 2012; 20:223-32.
9 Frischholz, S., et al., J. Biol. Chem., 1998; 273:4547.
10. Yamaguchi, N., et al. J. Biol. Chem., 1989; 264:16022.
11. Olsen, B. J., and Ninomiya, Y., in: “Guidebook to the Extracellular Matrix and Adhesion Proteins”, Kreis, T., and Vale, R. (eds.), Oxford University Press, Oxford, pp. 32-48 (1993).
12. Schmid, T. M., and Linsenmayer, T. F., in: “Structure and Function of Collagen Types”, Mayne, R., and Burgeson, R. E. (eds.), Academic Press Inc., pp. 223-259 (1987).
13. Rucklidge, G. J., et al., Matrix Biol., 1996; 15; 73.
14. Aigner, T., et al., Histochem. Cell Biol., 1997; 107; 435.
15. Girkontaite, I., et al., Matrix Biol., 1996; 15; 231.
16. Gerstenfeld, L. C. and F. D. Shapiro, Expression of bone-specific genes by hypertrophic chondrocytes: implication of the complex functions of the hypertrophic chondrocyte during endochondral bone development. J Cell Biochem, 1996; 62:1-9.
17. Wei, F., et al., Activation of Indian hedgehog promotes chondrocyte hypertrophy and upregulation of MMP-13 in human osteoarthritic cartilage. Osteoarthritis Cartilage, 2012; 20:755-63.
18. Dong, Y. F., et al., Wnt induction of chondrocyte hypertrophy through the Runx2 transcription factor. J Cell Physiol, 2006; 208:77-86.
19. Horner, A., et al., Immunolocalisation of vascular endothelial growth factor (VEGF) in human neonatal growth plate cartilage. J Anat, 1999; 194:519-24.
20. Tsuchiya, A., et al., Expression of mouse HtrA1 serine protease in normal bone and cartilage and its upregulation in joint cartilage damaged by experimental arthritis. Bone, 2005; 37:323-36.
21. Huebner, J. L., et al., Transglutaminase 2 is a marker of chondrocyte hypertrophy and osteoarthritis severity in the Hartley guinea pig model of knee OA. Osteoarthritis Cartilage, 2009; 17:1056-64.
22. Fitzgerald, J. B., et al., Shear- and compression-induced chondrocyte transcription requires MAPK activation in cartilage explants. J Biol Chem, 2008; 283:6735-43.
23. Goldring, M. B., et al., Defining the roles of inflammatory and anabolic cytokines in cartilage metabolism. Ann Rheum Dis, 2008; 67:75-82.
24. Gefter M L, Margulies D H, Scharff M D. A simple method for polyethylene glycol-promoted hybridization of mouse myeloma cells. Somatic Cell Genet., 1977; 3:231-6.
25. Bay-Jensen, A. C., et al., Enzyme-linked immunosorbent assay (ELISAs) for metalloproteinase derived type II collagen neoepitope, CIIM—increased serum CIIM in subjects with severe radiographic osteoarthritis. Clin Biochem, 2011; 44:423-9.

1: An antibody, wherein said antibody specifically reacts with an epitope of collagen type X alpha 1, said epitope being comprised in the NC1 domain C-terminal amino acid sequence SFSGFLVAPM-COOH (SEQ ID NO: 1).

2: The antibody as claimed in claim 1, wherein said antibody is a monoclonal antibody, or a polyclonal antibody, or an antibody fragment.

3: The antibody as claimed in claim 1, wherein said antibody binds to an epitope comprised in the NC1 domain C-terminal amino acid sequence SFSGFLVAPM (SEQ ID NO: 1) of human collagen type X alpha 1.

4: A method of immunoassay for detecting in a biological sample an epitope comprised in the NC1 domain C-terminal amino acid sequence SFSGFLVAPM-COOH (SEQ ID NO: 1) of collagen type X alpha 1, said method comprising contacting said biological sample comprising said epitope comprised in said NC1 domain C-terminal amino acid sequence SFSGFLVAPM-COOH (SEQ ID NO: 1) of collagen type X alpha 1 with an antibody as claimed in claim 1, and determining the amount of binding of said antibody.

5: The method of immunoassay as claimed in claim 4, wherein said method is used to quantify the amount of intact collagen type X alpha 1 and fragments thereof comprising the amino acid sequence SFSGFLVAPM (SEQ ID NO: 1) in biofluid.

6: The method of immunoassay as claimed in claim 5, wherein said biofluid is synovial fluid, serum or plasma.

7: The method of immunoassay as claimed in claim 4, wherein said immunoassay is a competition assay or a sandwich assay.

8: The method of immunoassay as claimed in claim 4, wherein said immunoassay is a radioimmunoassay or an enzyme-linked immunosorbent assay.

9: The method of immunoassay as claimed in claim 4, wherein said method further comprises correlating the quantity of intact collagen type X alpha 1 and fragments thereof comprising said amino acid sequence SFSGFLVAPM (SEQ ID NO: 1) determined by said method with standard disease samples of known disease severity to evaluate the severity of a disease associated with collagen type X alpha 1.

10: The method of immunoassay as claimed in claim 9, wherein said method further comprises correlating the quantity of intact collagen type X alpha 1 and fragments thereof comprising said amino acid sequence SFSGFLVAPM (SEQ ID NO: 1) determined by said method with standard osteoarthritis samples of known severity.

11: The method of immunoassay as claimed in claim 9, wherein said method further comprises correlating the quantity of intact collagen type X alpha 1 and fragments thereof comprising said amino acid sequence SFSGFLVAPM (SEQ ID NO: 1) determined by said method with standard Ankylosing Spondylitis samples of known severity.

12: An assay kit for determining the quantity of intact collagen type X alpha 1 and fragments thereof comprising the amino acid sequence SFSGFLVAPM (SEQ ID NO: 1) in a biological sample, said kit comprising an antibody as claimed in claim 1 and at least one of:
a streptavidin coated 96 well plate;
a biotinylated peptide Biotin-L-SFSGFLVAPM-COOH (SEQ ID NO: 2), wherein L is an optional linker;
a secondary antibody for use in a sandwich immunoassay;
a calibrator peptide comprising the sequence SFSGFLVAPM (SEQ ID NO: 1);
an antibody biotinylation kit;
an antibody HRP labeling kit;
an antibody radiolabeling kit; or
an assay visualization kit.
